(19) United States
(12) Patent Application Publication
(10) Pub. No.: US 2022/0215907 A1
(43) Pub. Date: Jul. 7, 2022

(54) RETRIEVAL METHOD, COMPUTER-READABLE RECORDING MEDIUM, AND RETRIEVAL DEVICE

(71) Applicant: FUJITSU LIMITED, Kawasaki-shi (JP)

(72) Inventor: Nobuyuki KATAE, Fuchu (JP)

(73) Assignee: FUJITSU LIMITED, Kawasaki-shi (JP)

(21) Appl. No.: 17/705,399

(22) Filed: Mar. 28, 2022

Related U.S. Application Data
(63) Continuation of application No. PCT/JP2019/042950, filed on Oct. 31, 2019.

(51) Int. Cl. G16C 20/80; G16C 20/20; G16C 20/70; G16C 20/10; G06K 9/62

(52) U.S. Cl. CPC G16C 20/80; G16C 20/20; G06K 9/6215; G16C 20/10; G16C 20/70

(57) ABSTRACT

A retrieval device specifies the chemical structure of a compound indicated by a compound name included in an input document. The retrieval device totalizes, for each substructure of the chemical structure, the number of substructures included in the input document. The retrieval device generates a substructure vector of the input document based on the substructure and the number. The retrieval device outputs one or more documents similar to the input document from a plurality of documents including a stored compound name based on comparison between the substructure vector of the input document and each substructure vector of the documents.

### CROSS-REFERENCE TO RELATED APPLICATION

This application is a continuation of International Application No. PCT/JP2019/042950, filed on Oct. 31, 2019, the entire contents of which are incorporated herein by reference.

### FIELD

The embodiments discussed herein are related to a retrieval method, a computer-readable recording medium, and a retrieval device.

### BACKGROUND

Conventionally, there is known the technology of expressing a document written in a natural language by a distributed expression vector and retrieving a document using the similarity between distributed expression vectors. Such a technology may be used, in literature search and research and development, to retrieve documents related to a search target or a research and development target among existing documents such as papers and patent publications. Conventional technology is described in Japanese Laid-open Patent Publication No. 2006-331245, for example.

### SUMMARY

According to an aspect of an embodiment, a computer specifies a chemical structure of a compound indicated by a compound name included in an input document. The computer totalizes, for each substructure of the chemical structure, number of substructures included in the input document. The computer generates a vector of the input document based on the substructure and the number. And the computer outputs one or more documents from a plurality of documents including a compound name stored in a storage unit based on comparison between the vector of the input document and a vector of each of the documents.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a block diagram illustrating a configuration example of a retrieval device;

FIG. 2 is a diagram for explaining a flow of the entire processing by the retrieval device;

FIG. 3 is a block diagram illustrating a configuration example of a substructure vector calculation unit;

FIG. 4 is a diagram illustrating an example of a substructure list;

FIG. 5 is a diagram illustrating an example of a method for calculating a substructure vector of a first document;

FIG. 6 is a diagram illustrating an example of a method for calculating a substructure vector of a second document;

FIG. 7 is a diagram illustrating an example of a method for calculating similarity of substructure vectors;

FIG. 8 is a diagram illustrating an example of a screen to be output;

FIG. 9 is a flowchart illustrating a flow of processing for constructing a document database;

FIG. 10 is a flowchart illustrating a flow of processing for retrieving a document;

FIG. 11 is a diagram illustrating an example of a method for calculating a substructure cooccurrence vector of the first document;

FIG. 12 is a diagram illustrating an example of a method for calculating a substructure cooccurrence vector of the second document;

FIG. 13 is a diagram illustrating an example of a method for calculating similarity of substructure cooccurrence vectors;

FIG. 14 is a diagram illustrating an example of a method for calculating weighted similarity of substructure vectors; and

FIG. 15 is a diagram for explaining a hardware configuration example.

### DESCRIPTION OF EMBODIMENTS

With the above-described technology, it may be difficult to retrieve a document in the chemical field with high accuracy. Documents in the chemical field frequently include the names of compounds related to materials, chemicals, or the like. Here, a compound name has a plurality of other names. In other words, one compound has several to several tens other compound names. Moreover, approximately one hundred million kinds of compound names exist.

Furthermore, to find a distributed expression vector of a compound name, a large amount of text data including the compound name should be collected. However, it is actually difficult to collect such text data and prepare an effective distributed expression vector.

Preferred embodiments of the present invention will be explained with reference to accompanying drawings. Note that the embodiments do not limit the invention. It is also possible to appropriately combine the embodiments in a range not causing any contradiction.

### [a] First Embodiment

Functional Configuration

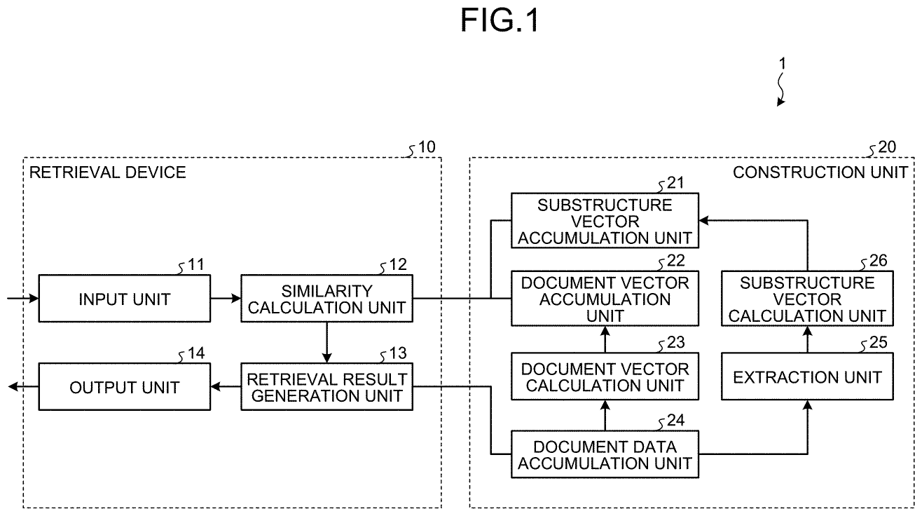

The following will describe a configuration of a retrieval device according to an embodiment with reference to FIG. 1. FIG. 1 is a block diagram illustrating a configuration example of a retrieval device. As illustrated in FIG. 1, a retrieval device 1 includes a retrieval unit 10 and a construction unit 20.

The retrieval unit 10 retrieves a document similar to an entered input document from a database including a plurality of documents. The construction unit 20 calculates a substructure vector of a document. The construction unit 20 also accumulates documents and substructure vectors. Furthermore, the construction unit 20 is able to calculate and accumulate not only substructure vectors but also document vectors.

Here, the document vector is a vector that expresses meaning of a document by a vector using a machine learning technique such as a neural network. With the document vector, it is possible to quantitatively evaluate the similarity of the meanings between documents. For example, the document vector is a distributed expression vector being a real numerical vector of about 50 to 300 dimensions. Note that the distributed expression is also referred to as “embedding”. Word2Vec, Doc2Vec, and the like are known as the technologies for calculating the distributed expression vector.

The substructure vector is a vector that expresses meaning of compounds in a document. Documents in the chemical field are characterized in the frequent appearance of compound names. Then, when the distributed expression vector is applied to a document in the chemical field, there is a case in which the retrieval with high accuracy is not achieved because each compound name has a plurality of other names. In addition, to improve the accuracy, an enormous number of text data of documents in the chemical field may be collected. However, it is actually difficult to collect such text data.

Meanwhile, the retrieval device 1 achieves the retrieval of documents in the chemical field with high accuracy with use of the substructure vector. Moreover, the retrieval device 1 is able to further improve the accuracy by performing the retrieval using both the substructure vector and the document vector. That is, because the document vector allows the semantic comparison between an input document and a plurality of documents, the retrieval device 1 is able to output a document from the plurality of documents on the basis of the comparison between the substructure vectors and the semantic comparison between the input document and the plurality of documents.

As illustrated in FIG. 1, the retrieval unit 10 includes an input unit 11, a similarity calculation unit 12, a retrieval result generation unit 13, and an output unit 14. An input document is entered to the input unit 11. The input document may be considered to be a query for retrieval or a generation origin of a query.

The similarity calculation unit 12 calculates similarity between an input document and other documents. To be more specific, the similarity calculation unit 12 calculates similarity between vectors that expresses the characteristics of documents and are calculated by the construction unit 20. The similarity calculation unit 12 is able to calculate, as the similarity, a distance between vectors, cosine similarity, and the like.

The retrieval result generation unit 13 generates data of a predetermined format representing a retrieval result on the basis of the calculated similarity. For example, the retrieval result generation unit 13 is able to generate a list of documents with the similarity equal to or larger than a threshold, or a list of a given number of documents arranged in the descending order of similarity. The output unit 14 outputs a retrieval result generated by the retrieval result generation unit 13. The output unit 14 may output a retrieval result as a file or by screen display.

The construction unit 20 includes a substructure vector accumulation unit 21, a document vector accumulation unit 22, a document vector calculation unit 23, a document data accumulation unit 24, an extraction unit 25, and a substructure vector calculation unit 26.

The document data accumulation unit 24 accumulates text data of documents. The document vector calculation unit 23 calculates a document vector. The document vector accumulation unit 22 accumulates document vectors. Note that the document here may be an input document or a document to be retrieved or output.

The extraction unit 25 extracts compound names from an input document and documents accumulated by the document data accumulation unit 24. For example, the extraction unit 25 extracts compound names included in a document among compound names described in a preliminarily prepared master. The master may be preliminarily prepared manually or automatically. Moreover, the master may include a part or all of the compound names that can be named by a rule such as IUPAC nomenclature (reference URL: https://ja.wikipedia.org/wiki/IUPAC%E5%91%BD%E5%90%8D%E6%B3%95). In the following description, it is assumed that the compound name refers to the chemically expressed substance name in general, and includes, for example, an element name.

The substructure vector calculation unit 26 calculates a substructure vector. The substructure vector accumulation unit 21 accumulates substructure vectors. It is assumed that the document data in the document data accumulation unit 24, the document vector in the document vector accumulation unit 22, and the substructure vector in the substructure vector accumulation unit 21 are mutually associated by a common ID or the like.

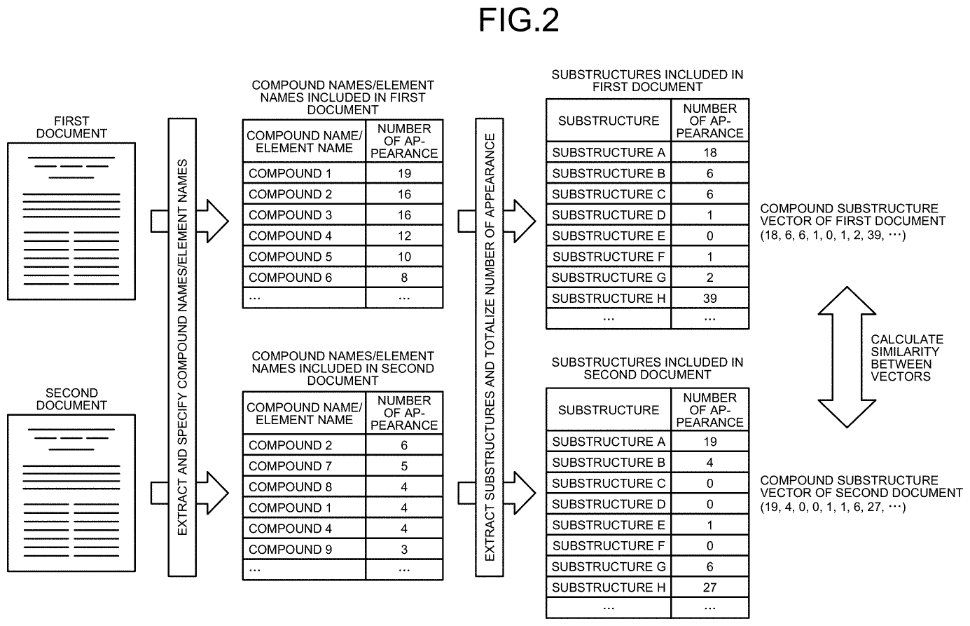

The following will describe a flow of entire processing by the retrieval device 1 with reference to FIG. 2. FIG. 2 is a diagram for explaining the flow of the entire processing by the retrieval device. The first document is an example of the input document. The second document is an example of the accumulated document. First, the retrieval device 1 extracts compound names and element names included in the first document and the second document to form a list of compound names/element names, and then extracts substructures and specifies the number of appearance of each substructure on the basis of the list of compound names/element names included in the first document and the second document. Then, the retrieval device 1 generates a substructure vector with the number of appearance of each substructure as a component of the vector.

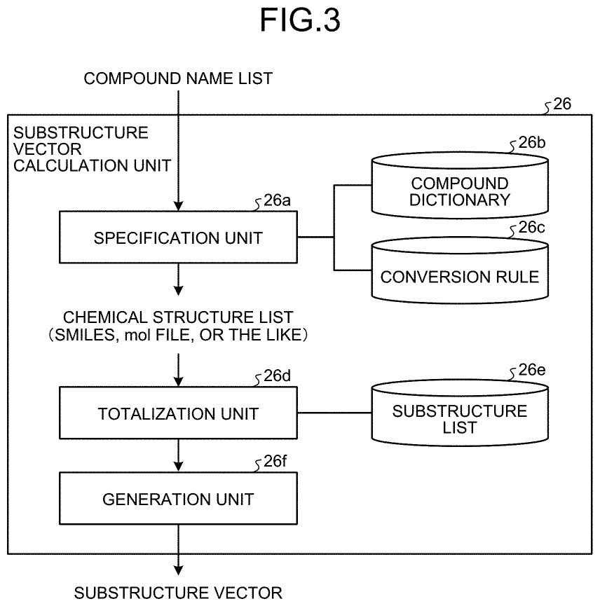

The substructure vector calculation unit 26 will be described in detail. FIG. 3 is a block diagram illustrating a configuration example of the substructure vector calculation unit. As illustrated in FIG. 3, the substructure vector calculation unit 26 includes a specification unit 26a, a compound dictionary 26b, a conversion rule 26c, a totalization unit 26d, a substructure list 26e, and a generation unit 26f. Furthermore, the substructure vector calculation unit 26 receives a compound name list that is a list of compound names extracted by the extraction unit 25, and outputs a substructure vector.

The specification unit 26a specifies the chemical structure of compounds indicated by compound names included in an input document. The specification unit 26a is able to specify the chemical structure of one compound indicated by a plurality of compound names described as other names, on the basis of the compound dictionary 26b and the conversion rule 26c. For example, the specification unit 26a is able to uniquely specify a compound by a chemical formula even in a case where a plurality of calling names exist.

The compound dictionary 26b is dictionary-form data in which a plurality of other names are associated to one chemical structure. For example, in the compound dictionary 26b, character strings such as “ethanol (written in Japanese letters)”, “ethyl alcohol (written in Japanese letters)”, “ethanol” “ethyl alcohol” “C₂H₆O”, “C₂H₅OH”, “CH₃CH₂OH”, “fermented alcohol”, and the like are associated to the chemical structure of ethanol. Furthermore, the conversion rule 26c is information indicating the rule of IUPAC nomenclature, and is information allowing the specification of the chemical structure of ethanol based on the character string “ethanol”.

The totalization unit 26d totalizes, for each substructure of a chemical structure, the number of the substructure included in an input document. The totalization unit 26d receives a chemical structure list from the specification unit 26a. The chemical structure list is, for example, SMILES or a mol file. The totalization unit 26d refers to the substructure list 26e to specify substructures of the chemical structures included in the chemical structure list, and totalizes the number of each substructure.

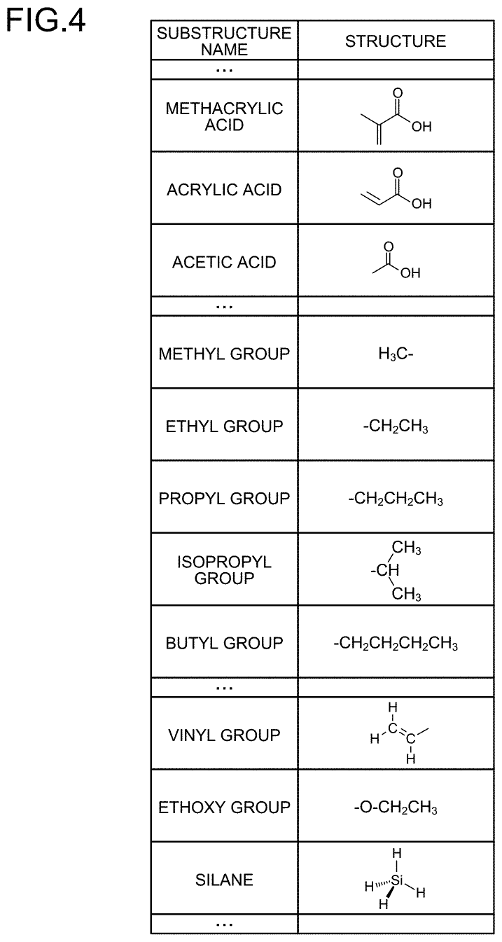

FIG. 4 is a diagram illustrating an example of the substructure list. As illustrated in FIG. 4, the substructure names and the structures are included in the substructure list 26e. For example, the substructure list 26e represents that the structure of the substructure having the substructure name “methyl group” is “H₃C—”.

The substructures include certain important main parts, substituents, and the like, such as primary, secondary, tertiary, and quaternary carbon, a hydroxy group, an amino group, an amide group, an imino group, a carboxy group, a thiol group, a benzene ring, and the like, in addition to those illustrated in the drawing.

The generation unit 26f generates a substructure vector of an input document on the basis of the substructures and the numbers thereof. The generation unit 26f generates a substructure vector with the number of each substructure as a component. Furthermore, the generation unit 26f may generate a substructure vector with the information indicating whether the number of each substructure is zero as a component. The information indicating whether the number of each substructure is zero is, for example, 0 or 1.

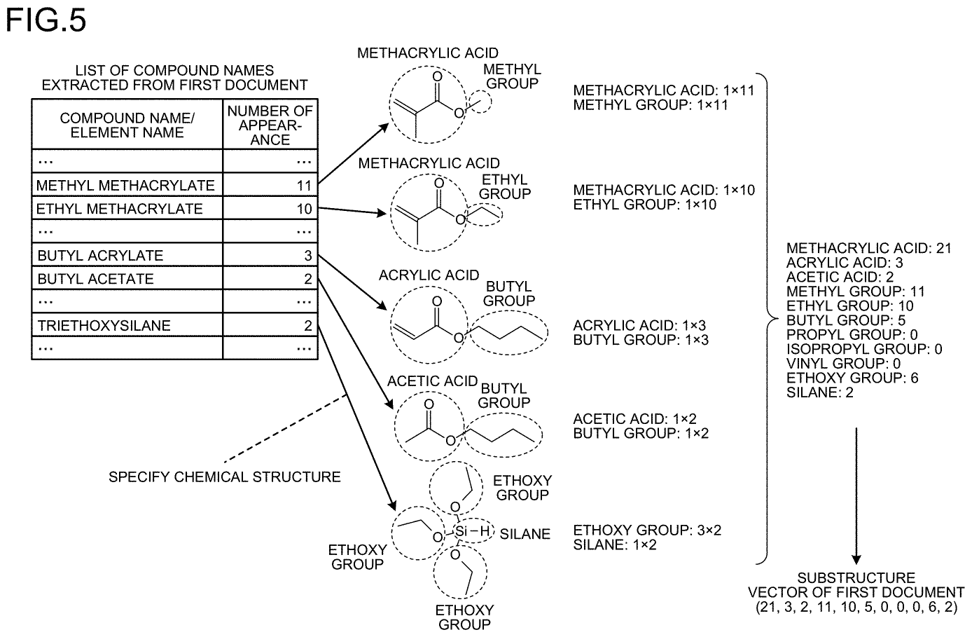

FIG. 5 is a diagram illustrating an example of a method for calculating a substructure vector of the first document. As illustrated in FIG. 5, the specification unit 26a first specifies a chemical structure from the compound name list. Then, the totalization unit 26d totalizes the number of substructures of the specified chemical structure.

Here, the totalization unit 26d is able to calculate the sum of the products between the number of each substructure included in each compound and the number of each compound name indicating the compound included in the input document, as the number of substructures included in the input document.

In the example of FIG. 5, the numbers of methacrylic acid and a methyl group, which are substructures of methyl methacrylate, are both one. Moreover, the number of appearance of methyl methacrylate included in the first document is 11. Furthermore, methacrylic acid is also a substructure of ethyl methacrylate. Then, the number of ethyl methacrylate included in the first document is 10. From this, the totalization unit 26d totalizes the number of methacrylic acid in the first document as 1×11+1×10=21.

Supposing that the compound list of the first document represents that the number of appearance of “methyl methacrylate” is 11 and the number of appearance of “C₅H₈O₂” is two, the totalization unit 26d totalizes the number of methyl methacrylate included in the first document as 11+2=13. Note that C₅H₈O₂ is the chemical formula of methyl methacrylate.

Furthermore, in the example of FIG. 5, the number of ethoxy groups that are substructures of triethoxysilane is three. The number of appearance of triethoxysilane included in the first document is two. From this, the totalization unit 26d totalizes the number of ethoxy groups in the first document as 3×2=6.

The generation unit 26f generates a substructure vector with the number totalized by the totalization unit 26d as a component. For example, the first component of the substructure vector is the number of methacrylic acid. Moreover, the second component of the substructure vector is the number of acrylic acid.

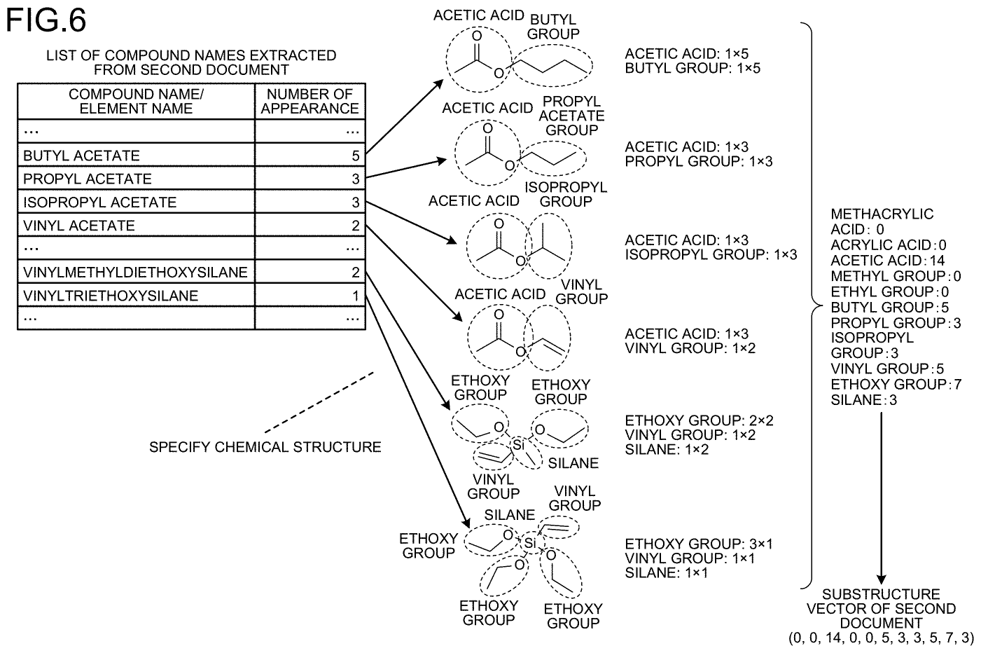

FIG. 6 is a diagram illustrating an example of a method for calculating a substructure vector of the second document. In the example of FIG. 6, two ethoxy groups are included in vinylmethyldiethoxysilane and three ethoxy groups are included in vinyltriethoxysilane. Furthermore, the numbers of vinylmethyldiethoxysilane and vinyltriethoxysilane included in the second document is two and one, respectively. From this, the totalization unit 26d totalizes the number of ethoxy groups in the second document as 2×2+3×1=7.

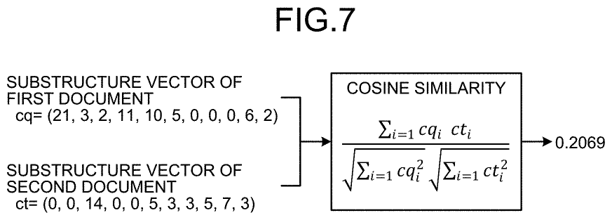

The similarity calculation unit 12 calculates similarity between the substructure vector of the first document and the substructure vector of the second document. FIG. 7 is a diagram illustrating an example of a method for calculating similarity of substructure vectors. As illustrated in FIG. 7, the similarity calculation unit 12 calculates cosine similarity between the substructure vector cq of the first document and the substructure vector ct of the second document as 0.20609. Note that the number of components of each substructure vector is equal to the number of the kinds of substructures in each document. For example, each of the first document and the second document includes 11 substructures in total without permitting overlapping. Thus, the number of the components of the substructure vector is 11.

Furthermore, the similarity calculation unit 12 may calculate a score that combines the similarity of substructure vectors and the similarity of document vectors. It is assumed that an input document as a query is D_{Q}, and a document to be retrieved is D_{T}. Then, the similarity calculation unit 12 calculates a similarity score Score(D_{Q}, D_{T}) as in the expression (1).

Score(D_{Q},D_{T})=Sim_{Emb})(D_{Q},D_{T})+Sim_{Chem}(D_{Q},D_{T}) (1)

Supposing that the document vectors of the document D_{Q} and the document D_{T} are E_{Q}=(eq₁, eq₂, . . . ) and E_{T}=(et₁, et₂, . . . ), respectively, the similarity calculation unit 12 calculates similarity sim_{Emb} of the document vectors and similarity sim_{Chem} of the substructure vectors as in the expressions (2) and (3).

$$\begin{matrix}{Sim}_{Emb} ( {D}_{Q} , {D}_{T} ) = cos ( {E}_{Q} , {E}_{T} ) = \frac{{\sum }_{i = 1} {eq}_{i} {et}_{i}}{\sqrt{{\sum }_{i = 1} {eq}_{i}^{2}} \sqrt{{\sum }_{i = 1} {et}_{i}^{2}}} & ( 2 ) \\ {Sim}_{Chem} ( {D}_{Q} , {D}_{T} ) = cos ( {C}_{Q} , {C}_{T} ) = \frac{{\sum }_{i = 1} {cq}_{i} {ct}_{i}}{\sqrt{{\sum }_{i = 1} {cq}_{i}^{2}} \sqrt{{\sum }_{i = 1} {ct}_{i}^{2}}} & ( 3 )\end{matrix}$$

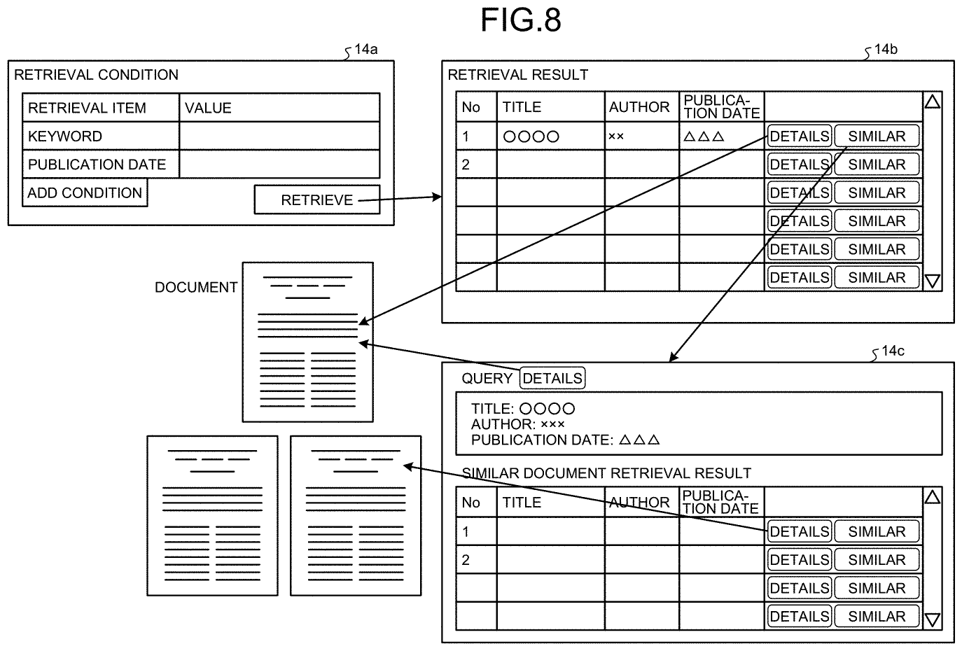

The output unit 14 is able to display a screen generated by the retrieval result generation unit 13. FIG. 8 is a diagram illustrating an example of a screen to be output. As illustrated in FIG. 8, the output unit 14 first displays a retrieval condition input screen 14a. In the retrieval condition input screen 14a, a retrieval condition such as a keyword and a publication data of a document are entered.

When a “RETREIVAL” button on the retrieval condition input screen 14a is pressed, the retrieval result generation unit 13 retrieves a document matching a retrieval condition from the document data accumulation unit 24. The retrieval here does not necessarily use a substructure vector, and may be mere retrieval of a document that includes a character string matching a keyword. Then, the output unit 14 displays a retrieval result display screen 14b.

When a “DETAILS” button on the retrieval result display screen 14b is pressed, the corresponding document data is downloaded. Furthermore, when a “SIMILAR” button on the retrieval result display screen 14b is pressed, the output unit 14 displays a list of documents similar to the corresponding document data on a similar document list screen 14c.

Here, the retrieval device 1 retrieves documents using a substructure vector with a document corresponding to the “SIMILAR” button on the retrieval result display screen 14b as an input document. Then, when a “DETAILS” button on the similar document list screen 14c is pressed, the corresponding document data is downloaded.

Furthermore, when the “SIMILAR” button on the similar document list screen 14c is pressed, the output unit 14 switches the similar document list screen 14c to display a list of documents similar to the corresponding document data.

That is, the similarity calculation unit 12 calculates similarity of the input document to each of a plurality of documents on the basis of the comparison between the vector of the input document and the vector of each document including a compound name stored in the storage unit. Then, the output unit 14 displays, on the display screen, a list of documents included in a plurality of documents in the descending order of the calculated similarity. The similar document list screen 14c is an example of a list displayed by the output unit 14.

Flow of Processing

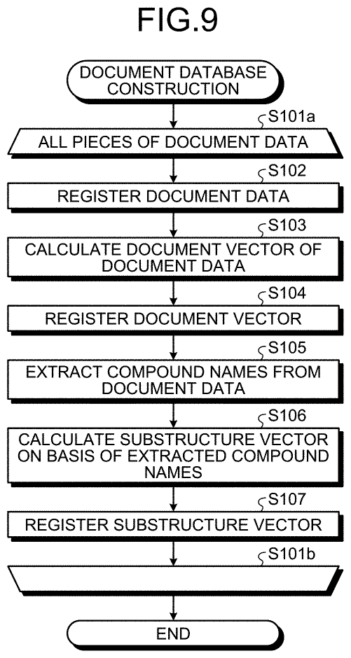

The following will describe processing for constructing a document database with reference to FIG. 9. FIG. 9 is a flowchart illustrating a flow of processing for constructing the document database. The document database is the document data accumulation unit 24, the document vector accumulation unit 22, and the substructure vector accumulation unit 21 of the construction unit 20. That is, the retrieval device 1 generates and stores document vectors and substructure vectors corresponding to document data by the processing for constructing a database.

First, the retrieval device 1 repeats the processing from S102 to S107 for each piece of the whole prepared document data (Steps S101a, S101b). First, as illustrated in FIG. 9, the retrieval device 1 registers document data in the document data accumulation unit 24 (Step S102).

Then, the retrieval device 1 calculates a document vector of the registered document data (Step S103), and registers the calculated document vector in the document vector accumulation unit 22 (Step S104).

Next, the retrieval device 1 extracts compound names from the registered document data (Step S105). Then, the retrieval device 1 calculates a substructure vector on the basis of the extracted compound names (Step S106), and registers the calculated substructure vector in the substructure vector accumulation unit 21 (Step S107).

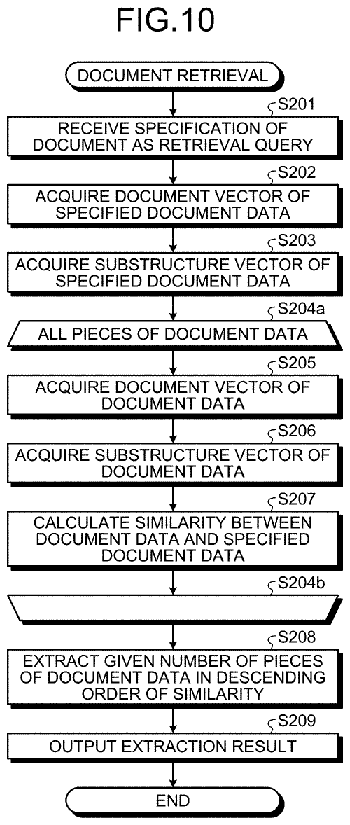

The following will describe the processing for retrieving documents with reference to FIG. 10. FIG. 10 is a flowchart illustrating a flow of the processing for retrieving documents. As illustrated in FIG. 10, the retrieval device 1 receives specification of a document as a retrieval query (Step S201). The specified document may be a newly input document or a document registered in the document database.

The retrieval device 1 acquires a document vector of the specified document data (Step S202). Then, the retrieval device 1 acquires a substructure vector of the specified document data (Step S203). The document vector and the substructure vector may be vectors registered in the document database or newly calculated vectors.

Here, the retrieval device 1 repeats the processing from steps S205 to S207 for each piece of the whole document data registered in the database (Steps S204a and S204b). As illustrated in FIG. 10, the retrieval device 1 first acquires a document vector of the document data (Step S205). Next, the retrieval device 1 acquires a substructure vector of the document data (Step S206). Then, the retrieval device 1 calculates similarity between the document data and the specified document data (Step S207).

The retrieval device 1 extracts a given number of pieces of document data in the descending order of similarity (Step S208). Then, the retrieval device 1 outputs an extraction result (step S209). For example, the retrieval device 1 outputs the result onto the similar document list screen 14c.

### Advantageous Effects

As described above, the specification unit 26a specifies the chemical structure of a compound indicated by a compound name included in an input document. The totalization unit 26d totalizes, for each substructure of a chemical structure, the number of the substructure included in the input document. The generation unit 26f generates a substructure vector of the input document on the basis of the substructures and the numbers thereof. Furthermore, the output unit 14 outputs a document from a plurality of documents on the basis of comparison between the substructure vector of the input document and the substructure vector of each of a plurality of documents including a compound name stored in the construction unit 20. In this manner, the retrieval device 1 is able to uniquely specify a compound even when the compound has a plurality of other names. The retrieval device 1 is also able to calculate a vector expressing the characteristics of a document in the chemical field without a large amount of document data. As a result, the retrieval device 1 is able to retrieve documents in the chemical field with high accuracy.

The generation unit 26f generates a substructure vector with the number of each substructure or the information indicating whether the number of each substructure is zero as a component. As a result, the retrieval device 1 is able to select a method for generating a substructure vector considering the accuracy and the calculation amount.

The totalization unit 26d calculates the sum of the products between the number of each substructure included in each compound and the number of each compound name indicating the compound included in the input document, as the number of the substructure included in the input document. In this manner, in the retrieval device 1, as the number of appearance of a substructure is larger and as the number of a substructure included in one compound is larger, a value of the component in a substructure vector is increased. In this manner, the retrieval device 1 is able to express the characteristics of substructures in the document more clearly.

The output unit 14 outputs documents from a plurality of documents on the basis of the comparison between substructure vectors and the semantic comparison between the input document and a plurality of documents. In this manner, the retrieval device 1 performs retrieval using both the document vector and the substructure vector, thereby further improving the accuracy.

The similarity calculation unit 12 calculates similarity of the input document to each of a plurality of documents on the basis of the comparison between the vector of the input document and each vector of the documents including a compound name stored in the storage unit. Then, the output unit 14 displays, on the display screen, a list of documents included in a plurality of documents in the descending order of the calculated similarity. Therefore, the user is able to easily grasp a list of documents similar to the input document.

### [b] Second Embodiment

The substructure vector may express the cooccurrence relation between substructures, in addition to the number of individual substructure. In this case, the totalization unit 26d further totalizes the number of each combination of substructures included in the input document. Furthermore, the generation unit 26f generates a substructure vector of the input document on the basis of both the number of each substructure and the number of each combination of substructures that are totalized by totalization processing. The substructure vector generated here is referred to as a substructure cooccurrence vector.

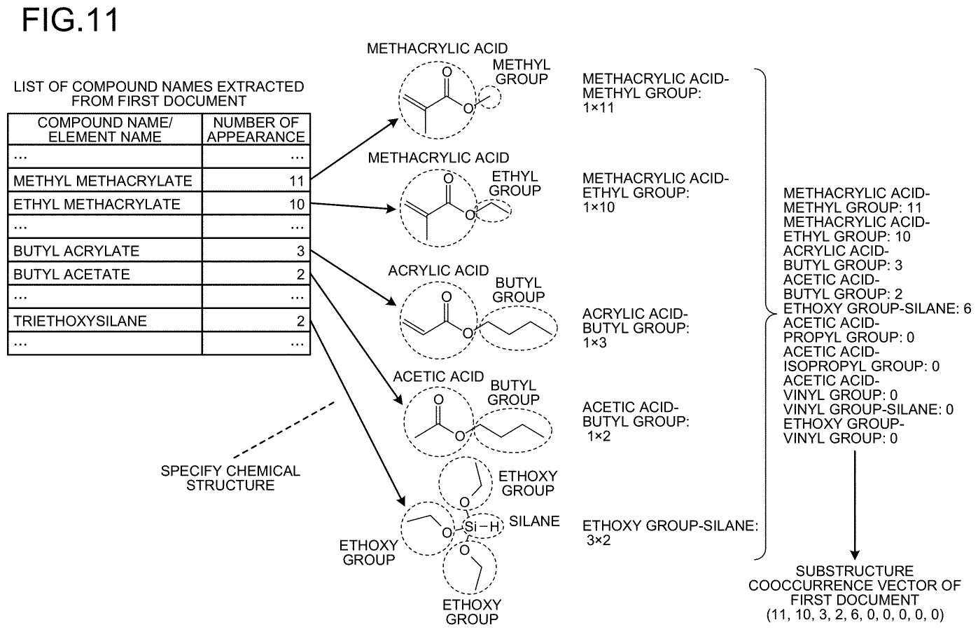

FIG. 11 is a diagram illustrating an example of a method for calculating the substructure cooccurrence vector of the first document. In the example of FIG. 11, methyl methacrylate includes one combination of a methacrylic acid and a methyl group. Furthermore, the number of methyl methacrylate included in the first document is 11. Here, the totalization unit 26d totalizes the number of the combinations of a methacrylic acid and a methyl group in the first document as 1×11=11. Similarly, the totalization unit 26d totalizes each combination of substructures.

The generation unit 26f generates a substructure vector with the number totalized by the totalization unit 26d as a component. In the example of FIG. 11, the first component of the substructure vector is the number of the combinations of methacrylic acid and a methyl group. The second component of the substructure vector is the number of the combinations of methacrylic acid and an ethyl group.

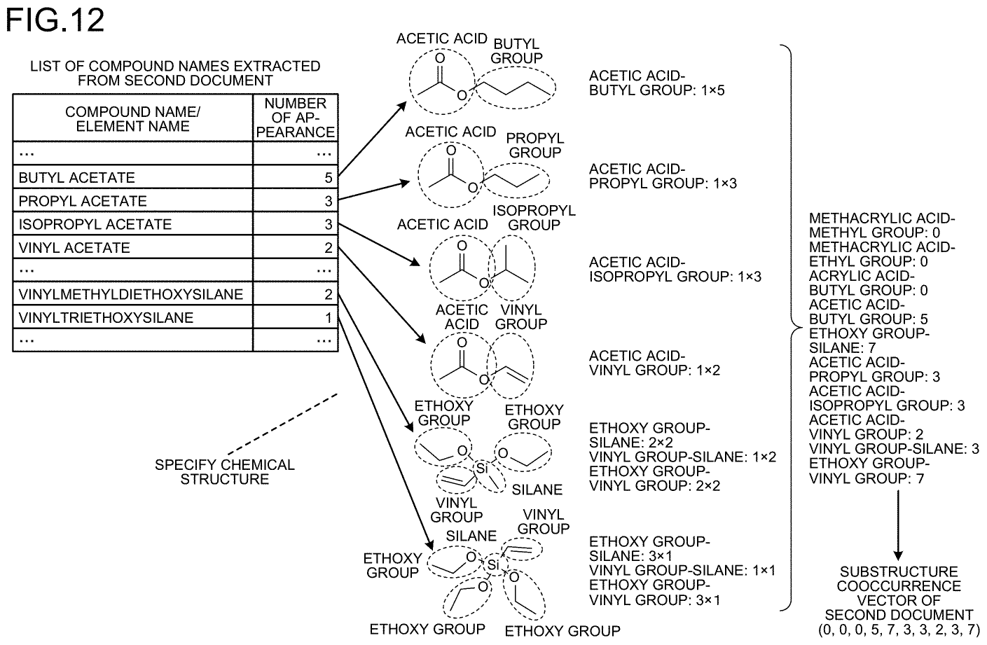

FIG. 12 is a diagram illustrating an example of a method for calculating a substructure cooccurrence vector of the second document. In the example of FIG. 12, vinylmethyldiethoxysilane includes two combinations of an ethoxy group and a silane. Moreover, vinyltriethoxysilane includes three combinations of an ethoxy group and a silane. The number of vinylmethyldiethoxysilanes included in the second document is two. The number of vinyltriethoxysilanes included in the second document is one. Here, the totalization unit 26d totalizes the number of combinations of an ethoxy group and a silane in the second document as 2×2+3×1=7.

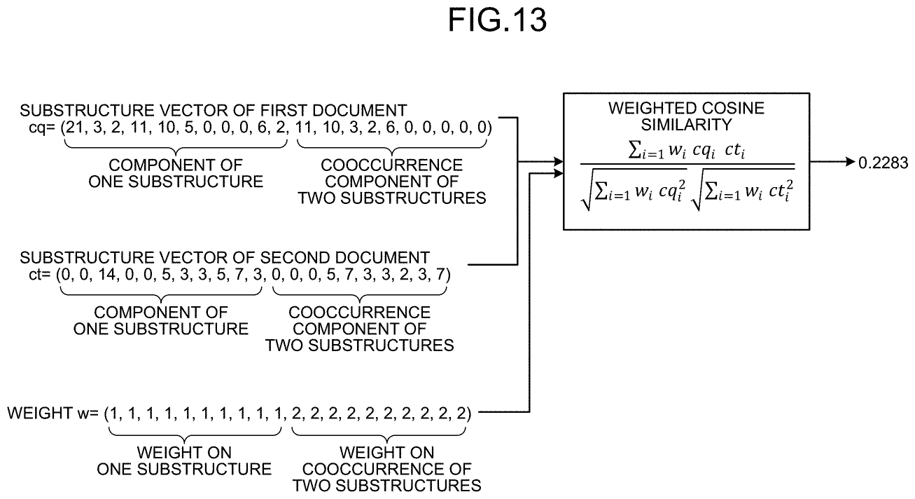

FIG. 13 is a diagram illustrating an example of a method for calculating similarity between substructure cooccurrence vectors. As illustrated in FIG. 13, the similarity calculation unit 12 calculates weighted cosine similarity between a vector cq formed by synthesizing the substructure vector and the substructure cooccurrence vector of the first document and a vector ct formed by synthesizing the substructure vector and the substructure cooccurrence vector of the second document as 0.2283. Here, the similarity calculation unit 12 multiplies the component of the substructure vector by a weight 1, and multiplies the component of the substructure cooccurrence vector by a weight 2, in the synthesized vectors.

Moreover, the retrieval device 1 may further totalize the number of combinations of three substructures and include a totalization result in the vector. In this case, the similarity calculation unit 12 may multiply the component representing the cooccurrence relation of three substructures by a weight 3.

It is assumed that an input document as a query is D_{Q}, and a document to be retrieved is D_{T}. Here, the similarity calculation unit 12 calculates a similarity score Score(D_{Q}, D_{T}) as in the expression (4).

Score(D_{Q},D_{T})=Sim_{Emb})(D_{Q},D_{T})+Sim_{Chem}(D_{Q},D_{T}) (4)

Assuming that the substructure vectors of the document D_{Q} and the document D_{T} are C_{Q}=(cq₁, cq₂, . . . ) and C_{T}=(ct₁, ct₂, . . . ), respectively, and the weight is W=(w₁, w₂, the similarity calculation unit 12 calculates similarity sim_{Chem2} between the substructures vector as in the expression (5).

$${Sim}_{Chem 2} ( {D}_{Q} , {D}_{T} ) = \frac{{\sum }_{i = 1} {w}_{i} {cq}_{i} {ct}_{i}}{\sqrt{{\sum }_{i = 1} {w}_{i} {cq}_{i}^{2}} \sqrt{{\sum }_{i = 1} {w}_{i} {ct}_{i}^{2}}} \qquad ( 5 )$$

The cooccurrence relation of substructures may determine the characteristics of a compound. For this reason, in the second embodiment, the cooccurrence relation is considered, thereby retrieving semantically more similar documents.

### [c] Third Embodiment

The retrieval device 1 may calculate similarity after weighting each substructure on the basis of the appearance frequency. In this case, the output unit 14 outputs documents from a plurality of documents on the basis of the comparison between a vector generated by weighting a vector generated by generation processing on the basis of the given appearance frequency of a substructure in documents and each vector of the plurality of documents.

The weight based on the appearance frequency is, for example, an inverse document frequency (idf). Assuming that N is the total number of documents and df(t) is the number of documents including the substructure t, the idf is calculated as idf(t)=log(N/df(t))+1.

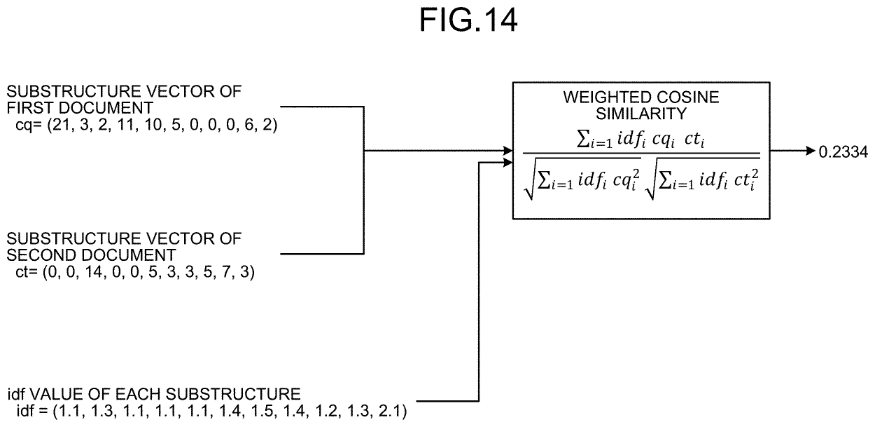

FIG. 14 is a diagram illustrating an example of a method for calculating weighted similarity of substructure vectors. As illustrated in FIG. 14, the similarity calculation unit 12 calculates weighted cosine similarity between the substructure vector cq of the first document and the substructure vector ct of the second document as 0.2334. Here, the similarity calculation unit 12 uses an idf value of each substructure as a weight.

Assuming that the substructure vectors of the document D_{Q} and the document D_{T} are C_{Q}=(cq₁, cq₂, . . . ) and C_{T}=(ct₁, ct₂, . . . ), respectively, and the weight based on the appearance frequency of each substructure is IDF=(idf₁, idf₂, . . . ), the similarity calculation unit 12 calculates a similarity score as in the expression (6). Furthermore, the similarity calculation unit 12 calculates similarity sim_{Chem3} of substructure vectors as in the expression (7).

$$\begin{matrix}Score ( {D}_{Q} , {D}_{T} ) = {Sim}_{Emb} ( {D}_{Q} , {D}_{T} ) + {Sim}_{chem 3} ( {D}_{Q} , {D}_{T} ) & ( 6 ) \\ {Sim}_{Chem 3} ( {D}_{Q} , {D}_{T} ) = \frac{{\sum }_{i = 1} {id}_{i} {fcq}_{i} {ct}_{i}}{\sqrt{{\sum }_{i = 1} {id}_{i} {fcq}_{i}^{2}} \sqrt{{\sum }_{i = 1} {id}_{i} {fct}_{i}^{2}}} & ( 7 )\end{matrix}$$

For example, a substructure appearing with low frequency throughout a whole document database, such as silane, has an important implication when it is included in the document, and may have a significant influence on the calculation of similarity. For this reason, in the third embodiment, the appearance frequency is considered, thereby retrieving semantically more similar documents.

Note that the retrieval device 1 may calculate the similarity by adding both the weight of the second embodiment and the weight of the third embodiment. In this case, each component of the substructure cooccurrence vector is multiplied by both a weight based on cooccurrence and a weight based on the appearance frequency of each combination, for example.

System

The information including processing procedures, control procedures, concrete names, various kinds of data and parameters in the above description and drawings may be arbitrarily changed unless otherwise specified. Moreover, the concrete examples, distributions, numerical values, and the like described in the embodiments are merely examples, and may be changed arbitrarily.

Furthermore, the components of the illustrated devices are function conceptual, and do not necessarily need to be physically configured as illustrated in the drawings. That is, the concrete form of distribution and integration of the devices is not limited to those illustrated in the drawings. That is, it is possible to configure all or a part thereof to be functionally or physically distributed and integrated in an arbitrary unit in accordance with various loads, usage conditions, and the like. Furthermore, all or an arbitral part of the processing functions performed in the devices may be realized by a central processing unit (CPU) and a program analyzed and executed by the CPU, or may be realized as hardware by wired logic.

Hardware

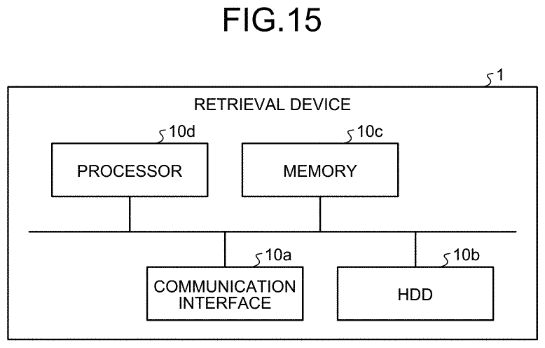

FIG. 15 is a diagram for explaining a hardware configuration example. As illustrated in FIG. 15, the retrieval device 1 includes a communication interface 10a, a hard disk drive (HDD) 10b, a memory 10c, and a processor 10d. The components illustrated in FIG. 15 are mutually connected by a bus or the like.

The communication interface 10a is a network interface card or the like, and performs communication with other servers. The HDD 10b stores programs and DBs that operate the functions illustrated in FIG. 2.

The processor 10d is a hardware circuit that operates a process for executing each function described in FIG. 1 and the like by reading out a program executing the same processing as the processing units illustrated in FIG. 1 from the HDD 10b or the like and loading the program in the memory 10c. That is, this process executes the same functions as the processing units of the retrieval device 1. To be more specific, the processor 10d reads out a program having the same functions as the retrieval unit 10 and the construction unit 20 from the HDD 10b or the like. Then, the processor 10d executes a process for performing the same processing as the retrieval unit 10, the construction unit 20, and the like.

In this manner, the retrieval device 1 operates as an information processing device performing a retrieval method by reading out and executing a program. Moreover, the retrieval device 1 is able to achieve the same functions as those in the above-described embodiments by reading out the program from a recording medium by a media reader and executing the read-out program. Note that a program in other embodiments is not limited to being executed by the retrieval device 1. For example, it is possible to similarly apply the invention when another computer or server executes a program or when they cooperate to execute a program.

This program may be distributed through a network such as the Internet. In addition, this program may be recorded on a computer-readable recording medium such as a hard disk, a flexible disk (FD), a CD-ROM, a magneto-optical disk (MO), and a digital versatile disc (DVD), and executed by being read out from the recording medium by a computer.

In one aspect of an embodiment of the invention, it is possible to retrieve a document in the chemical field with high accuracy.

All examples and conditional language recited herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although the embodiments of the present invention have been described in detail, it should be understood that the various changes, substitutions, and alterations could be made hereto without departing from the spirit and scope of the invention.

What is claimed is:

1. A retrieval method performed by a computer, the retrieval method comprising:
specifying a chemical structure of a compound indicated by a compound name included in an input document;
totalizing, for each substructure of the chemical structure, number of substructures included in the input document;
generating a vector of the input document based on the substructure and the number; and
outputting one or more documents from a plurality of documents including a compound name stored in a storage unit based on comparison between the vector of the input document and a vector of each of the documents.

2. The retrieval method according to claim 1, wherein the generating includes generating the vector including the number of each of the substructures or information indicating whether the number of each of the substructures is zero as a component of the vector.

3. The retrieval method according to claim 1, wherein
the totalizing includes further totalizing number of each combination of the substructures included in the input document, and
the generating includes generating the vector of the input document based on both the number of each of the substructures and the number of each combination of the substructures that are totalized at the totalizing.

4. The retrieval method according to claim 1, wherein the totalizing includes calculating a sum of products between the number of each of the substructures included in each of compounds and number of each of compound names indicating the compounds included in the input document, as number of each of the substructures included in the input document.

5. The retrieval method according to claim 1, wherein the outputting includes outputting a document from the documents based on comparison between a vector generated by weighting a vector generated at the generating based on given appearance frequency of each of the substructures in a document and the vector of each of the documents.

6. The retrieval method according to claim 1, wherein the outputting includes outputting a document from the documents based on comparison between the vectors and semantic comparison between the input document and the documents.

7. The retrieval method according to claim 1, wherein
the outputting includes
calculating similarity of the input document to each of the documents including a compound name stored in the storage unit based on comparison between the vector of the input document and the vector of each of the documents, and
displaying, on a display screen, a list of documents included in the documents in a descending order of the calculated similarity.

8. A non-transitory computer-readable recording medium having stored therein a program that causes a computer to execute a process comprising:
specifying a chemical structure of a compound indicated by a compound name included in an input document;
totalizing, for each substructure of the chemical structure, number of substructures included in the input document;
generating a vector of the input document based on the substructure and the number; and
outputting one or more documents from a plurality of documents including a compound name stored in a storage unit based on comparison between the vector of the input document and a vector of each of the documents.

9. A retrieval device, comprising:
a memory; and
a processor coupled to the memory, the processor being configured to execute a process including:
specifying a chemical structure of a compound indicated by a compound name included in an input document;
totalizing, for each substructure of the chemical structure, number of substructures included in the input document;
generating a vector of the input document based on the substructure and the number; and
outputting one or more documents from a plurality of documents including a compound name stored in a storage unit based on comparison between the vector of the input document and a vector of each of the documents.
